(19) United States
(12) Patent Application Publication
(10) Pub. No.: US 2025/0269342 A1
(43) Pub. Date: Aug. 28, 2025

(54) METHOD AND APPARATUS FOR PRODUCING A CALIBRATION MOISTURE-GAS MIXTURE

(71) Applicant: INSIGHT ANALYTICAL SOLUTIONS INC, Calgary (CA)

(72) Inventors: Kevin Harris, Calgary (CA); Philip C. Harris, Calgary (CA)

(21) Appl. No.: 19/209,378

(22) Filed: May 15, 2025

Related U.S. Application Data
(63) Continuation-in-part of application No. PCT/CA2023/051530, filed on Nov. 15, 2023.
(60) Provisional application No. 63/425,476, filed on Nov. 15, 2022.

(51) Int. Cl. B01J 7/00

(52) U.S. Cl. CPC B01J 7/00; B01J 2219/00051; B01J 2219/00162; B01J 2219/00164; B01J 2219/00227; B01J 2219/00961; B01J 2219/00963

(57) ABSTRACT

A method for converting a gas stream into a calibration moisture-gas mixture, which involves dividing a pressurised gas stream into first and a second portion streams, and saturating the first portion stream with water vapor at known and/or target pressure and at a known temperature to form a water-saturated gas stream. The flows of the water-saturated gas stream and the second portion stream are controlled to achieve a constant predefined flow ratio between the second portion stream and the water-saturated gas stream, and the two streams are mixed to form calibration moisture-gas mixture with a constant pre-defined dilution factor to obtain the calibration moisture-gas mixture with a desired/target moisture content. Also provided is an apparatus for converting the gas stream into the calibration moisture-gas mixture.

### CROSS-REFERENCE TO RELATED APPLICATIONS

This application is a continuation-in-part of International Application No. PCT/CA2023/051530, filed Nov. 15, 2023, which claims the benefit of priority to U.S. Provisional Patent Application No. 63/425,476, filed Nov. 15, 2022, the contents of both of which are hereby incorporated by reference in their entirety.

### FIELD

The present disclosure pertains to the field of analytical analyzers, and more particularly to the calibration of gas analyzers for measuring moisture content in a gas.

### BACKGROUND

One of the most common measurements made with gas analyzers is of the content of water vapor, moisture, or humidity in gases for various industries. The measurements may, for example, be used towards food storage, weather forecasting, chemical production, agriculture, semiconductor manufacturing, and oil and gas production. Commercially available analyzers need to be accurately calibrated to be useful.

Manufacturers of moisture analyzers calibrate new devices during manufacturing using equipment designed to generate known and controlled concentrations of water vapor in a gas stream. The equipment is typically purchased or specifically built for this purpose. Calibration usually involves adding water vapor to a dry gas stream either by permeation through a membrane or by saturation of the gas at a controlled temperature and pressure. Another common method, called the two-pressure method, involves saturating a gas at a high pressure and then dropping the pressure, which does not change the moisture content but lowers the relative humidity from 100% to a lower value.

These systems to generate moisture calibration standards are often complex and expensive—examples of commercial systems include Kin-Tek (accessible at kin-tek.com/span-pac-h20-system/), Michell (accessible at www.processsensing.com/en-us/products/optical-humidity-temperature-calibrator.htm), Fluke (accessible at us.flukecal.com/products/humidity-calibration#:˜:text=Humidity%20sensor%20calibration%20is%20typically,and%20divide d%20into%20two%20parts), Vaisala (accessible at www.vaisala.com/en/products/instruments-sensors-and-other-measurement-devices/instruments-industrial-measurements/hmk15), MBW Calibration (accessible at mbw.ch/products/humidity-generation/2500-generator/).

Most of these systems are geared towards calibrating relative humidity sensors or chilled mirror hydrometers that operate at or near atmospheric pressure in air and that are more suitable for use in a laboratory setting rather than rugged industrial locations. These relative humidity measurement systems are further designed for detecting fairly high levels of water vapor concentration (typically from thousands of ppm to a few percent by volume), which are much higher than the maximum values in semiconductor and natural gas industries.

Applications in industrial field locations requiring calibration for validation of a moisture analyzer accuracy sometimes use moisture calibration standard gas cylinders which are produced by gas cylinder manufactures such as Air Liquide and Scott (accessible at industry.airliquide.us/water-moisture-standards); however, these standard gas cylinders are expensive, and have limited accuracy and stability. If the moisture analyzers are installed outside of a building in a heated enclosure in a cold climate, an additional heated enclosure or jacket will be required for the cylinder, along with a heat-traced tubing bundle.

Some of the limitations of the commercially available moisture generators include large size of equipment, high cost, poor accuracy, instability, requirement for gas cylinders, and insufficient gas flow and delivery pressure. The equipment also needs supplies of electrical power that are problematic for gas plants, metering stations and other locations, all of which require all equipment to be certified for use in explosive atmospheres.

In addition, most of the presently known apparatuses or systems cannot generate moisture contents that are low enough to be suitable for the measurement ranges of natural gas moisture analyzers, and are also not compatible with using natural gas as the feed gas for producing the calibration gas flow. Most cannot deliver sufficient calibration gas and flow rate to make them compatible with natural gas moisture and water dew point analyzers.

Some systems that can produce low moisture concentrations typically rely on blending two gas streams using electronic mass flow controllers that require periodic recalibration and that are not compatible with varying and unknown gas compositions.

Therefore, there is a need for a simple and cost-effective method and apparatus for generating a calibration moisture-gas mixture accurately for natural gas and other gas applications.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

According to one example, the present disclosure is directed to a method and apparatus for producing a calibration moisture-gas mixture.

In accordance with an aspect of the present disclosure, there is provided a method for producing a calibration moisture-gas mixture for validation testing of moisture analyzers. The method comprises: receiving a pressurized gas stream from a gas supply and monitoring the pressure thereof; separating the pressurized gas stream into a first portion stream and a second portion stream; saturating the first portion stream with water vapor to form a water-saturated gas stream, at a known and/or a target pressure and a known temperature; controlling flow rate of the water-saturated gas stream and the second portion stream to achieve a constant predefined flow rate ratio between the second portion stream and the water-saturated gas stream; and mixing the water saturated-gas stream and the second portion stream to form the calibration moisture-gas mixture with a constant predefined dilution factor to obtain the calibration moisture-gas mixture with a target moisture content.

In accordance with another aspect of the present disclosure, there is provided an apparatus for producing a calibration moisture-gas mixture for validation testing of moisture analyzers. The apparatus comprises: an inlet configured to receive a pressurized gas stream from a gas supply; a pressure indicator to monitor pressure of the pressurized gas stream from the gas supply; a splitter configured to separate the pressurized gas stream into a first portion stream and a second portion stream; a saturation chamber configured to receive the first portion stream, the saturation chamber containing a water component for generating water vapor for saturating the first portion stream at a known and/or a target pressure and a known temperature to form a water-saturated gas stream; a temperature sensor configured to monitor temperature of the saturation chamber; a first flow control device positioned upstream or downstream of the saturation chamber and configured to regulate flow of the water-saturated gas stream flowing through/out from the saturation chamber; a second flow control device positioned downstream of the splitter and configured to regulate flow of the second portion stream; a mixing chamber configured to receive and mix the water-saturated gas stream and the second portion stream to form the calibration moisture-gas mixture; and an outlet configured to output the calibration moisture-gas mixture; wherein the first control device and second flow control device are configured to achieve a known constant flow ratio between the second portion stream and the water-saturated gas stream to provide a constant predefined dilution factor to form the calibration moisture-gas mixture with a desired moisture content.

### BRIEF DESCRIPTION OF THE FIGURES

The present disclosure will now be described by way of an exemplary embodiment with reference to the accompanying simplified, diagrammatic, not-to-scale drawings. In the drawings:

FIG. 1 depicts a simplified view of an apparatus for producing a calibration moisture-gas mixture, in accordance with an embodiment of the present disclosure.

### DETAILED DESCRIPTION

Unless defined otherwise, all technical and scientific terms used herein have the same meaning as commonly understood by one of ordinary skill in the art to which this invention belongs.

Unless the context requires otherwise, throughout this specification and claims, the words “comprise”, “comprising” and the like are to be construed in an open, inclusive sense.

The articles “a” and “an” are used herein to refer to one or to more than one (i.e., to at least one) of the grammatical object of the article. By way of example, “an element” means one element or more than one element.

As used herein, the term “about” refers to approximately a +/−10% variation from a given value. It is to be understood that such a variation is always included in any given value provided herein, whether or not it is specifically referred to.

The present disclosure provides methods and apparatus for generating calibration moisture-gas mixtures. The calibration moisture-gas mixtures may be used for calibrating moisture analyzers that are used for measuring water content and/or water dew point temperature in natural gas or other gases. The generation of the calibration moisture-gas mixture involves controlling flows of a wet/water-saturated gas stream and a second optionally dry gas stream to achieve a constant blend ratio, which can be stable for a long time and is independent of which gas is employed.

Methods of the present disclosure involve adjusting the moisture content of a gas by changing the saturation pressure rather than the saturation temperature, which causes almost instantaneous responses because changes in pressure typically stabilize faster than changes in temperature. The accuracy of the methods of the present disclosure is not affected by the operating pressure of the moisture analyzer that is to be calibrated, which can be high or low or unsteady.

The methods of the present disclosure can be conducted via a small and ruggedly designed apparatuses that are also light, portable, and resistant to damage and vibration. The Apparatuses of the present disclosure do not require use of expensive components like mass flow controllers and therefore are cost-effective.

The methods and apparatuses of the present disclosure do not require electrical components and are therefore suitable to be used in explosive gas atmospheres, such as those in natural gas metering stations and other locations, without additional safety certifications.

The methods of the present disclosure to generate the calibration moisture-gas mixtures can be conducted with high-pressure natural gas so that moisture analyzers can be calibrated with the actual natural gas that they are analyzing, which helps determine if the natural gas composition is affecting their accuracy.

The methods of the present disclosure can involve using either natural gas or gas from cylinders as the calibration gas media. The methods can further involve delivering flow rates up to 15 LPM and higher if required, and pressures up to half of the supplied natural gas pressure. This would be especially applicable for natural gas applications where the natural gas can be used as the high-pressure gas supply to the system.

The portable, rugged, and cost-effective apparatus for generating calibration moisture-gas mixtures of the present disclosure would not require utilities except for a high pressure gas supply to produce a gas mixture with adjustable water vapor content at a known level and with reasonable accuracy. The apparatus could be capable of delivering water vapor concentrations from close to zero and up to 150 ppm or higher as required, at a delivery pressure and flow rate suitable for checking calibration on a wide variety of analyzers used to measure moisture content and water dew point temperature of natural gas and other gases.

In one aspect, the present disclosure provides a method of producing a calibration moisture-gas mixture for validation testing of moisture analyzers. The method comprises the steps of receiving a pressurized gas stream from a gas supply and separating the same stream into a first portion stream and a second portion stream. The first portion stream is saturated with water vapor at a known and/or a target/desired pressure and at a known temperature to form a water-saturated gas stream. The flow rate of the water-saturated gas stream and the flow rate of the second portion stream is controlled to achieve a constant predefined flow ratio between the second portion stream and the water-saturated gas stream, and the two streams are mixed to form calibration moisture-gas mixture with a constant pre-defined dilution factor to obtain the calibration moisture-gas mixture with a desired/target moisture content.

In some embodiments, method also comprises regulating pressure of the pressurized gas stream before the separating step. In some embodiments, the pressure of the pressurized gas stream is regulated by an electronic pressure controller.

Alternatively, the method can also involve regulating the pressure of the water-saturated gas stream and the second portion stream to a desired/target value.

In some embodiments, the method further involves drying the second portion stream to form a dry-gas stream. In these embodiments, the flow of the second portion stream can be controlled before and/or after the drying step.

In some embodiments, the flow ratio between the second portion stream and the water-saturated gas stream is at least 1.5:1.

In some embodiments, the method involves using natural gas at a pipeline pressure as the pressurized gas stream. Other gases, such as carbon dioxide, hydrogen, nitrogen, sour gas, or mixtures of these gases, are also suitable for use as the pressurized gas stream if they are in a gas phase state at the delivery pressure to the inlet of the apparatus and the ambient temperature at the apparatus location.

The minimum pressure of the high-pressure supply depends on the desired/target delivery pressure for the calibrated moisture-gas mixture generated during the method and the desired adjustability of moisture content in the calibrated moisture-gas mixture can at least partly determine the minimum pressure.

In some embodiments, the method comprises regulating the pressure of the pressurized gas stream to be at least about 2.0 times the maximum desired outlet pressure of the calibrated moisture-gas mixture.

In some embodiments, the ratio between the inlet gas supply pressure and the outlet pressure of the moisture-gas mixture is at least 1.3, depending on the type of supply gas.

In some embodiments, the ratio between the inlet gas supply pressure and the outlet pressure of the moisture-gas mixture is about 1.5 to about 2.1.

The first portion stream is passed through a saturation chamber comprising a water component to form the water-saturated gas stream. Non limiting examples of suitable absorbent material include a sponge, a cotton wool, a cellulose fiber, etc.

In some embodiments, the second portion stream is passed through a drying chamber comprising a desiccant. Non-limiting examples of the desiccant include molecular sieve, alumina, phosphorus pentoxide, silica gel, etc.

In some embodiments, the flow rate of the water-saturated gas stream and/or the flow rate of the dry-gas stream is controlled using at least one of a flow orifice, a capillary, or a porous metal device. In some embodiments, the flow orifice is a critical flow orifice.

In another aspect, the present disclosure provides an apparatus for producing a calibration moisture-gas mixture for validation testing of moisture analyzers. The apparatus comprises an inlet configured to receive a pressurized gas stream from a gas supply, and a splitter configured to separate the pressurized gas stream into a first portion stream and a second portion stream. A pressure indicator is provided to monitor the pressure of the pressurized gas stream.

The apparatus further comprises a saturation chamber containing a water component and configured to receive the first portion stream of the pressurized gas stream to form a water-saturated gas stream. A first flow control device is positioned upstream or downstream of the saturation chamber and configured to regulate the flow of the water-saturated gas stream flowing through and/or out from the saturation chamber. A second flow control device is provided to regulate the flow of the second portion stream. A mixing member/chamber is provided to receive and mix the water-saturated gas stream and the second portion stream to form the calibration moisture-gas mixture, and an outlet is provided downstream of the mixing member to output the calibration moisture-gas mixture.

The first and second flow control devices are configured to achieve a known constant flow ratio between the second portion stream and the water-saturated gas stream to provide a constant pre-defined dilution factor to form the calibration moisture-gas mixture with a desired moisture content.

The dilution factor DF (expressed as 1:DF) can be calculated using the following equation:

$$DF = ( FO 1 + FO 2 ) / FO 1 \qquad Eq . 1$$

Where:
FO1 is the flow rate of the water-saturated gas stream through the first control device,
FO2 is the flow rate of the second portion stream through the second control device.

The flow rates for the first and second flow control devices can be calculated or measured by using either measurements of a reference gas at a reference inlet pressure, measurements by the flow control device manufacturer, or measurements for the actual gas used in the application.

Non-limiting examples of the first and second flow control devices include flow orifices, capillaries, and sintered or porous metal flow restrictors. In some embodiments, critical flow orifices are used as the first and second flow control devices.

The apparatus optionally comprises a pressure regulator upstream of the splitter to control water saturator contact pressure, the produced water vapor content, and/or the total system flow rate.

The apparatus optionally comprises a check valve upstream of the saturation chamber to prevent or minimize back-flow or diffusion of moisture from the saturation chamber to the inlet of the apparatus and/or into the pressurized/dry gas flow. The apparatus is optionally further provided with a back-pressure regulator and a pressure indicator positioned between the mixing member and the outlet, to monitor and regulate the pressure of the calibration moisture-gas mixture output at the outlet. Optionally, a vent member is connected to the back-pressure regulator to vent excess gas flow exceeding flow capacity of the moisture analyzer being tested (i.e., a “moisture analyzer flow capacity”).

In some embodiments, the apparatus further comprises a drying chamber configured to receive the second portion stream to form a dry-gas stream. The second flow control device can be positioned upstream or downstream of the drying chamber. The second flow device can be configured to regulate the flow of the dry-gas stream.

In some embodiments, a selection/shut-off valve can be provided downstream of the first flow control device, and selection/shut-off valves can be provided upstream and/or downstream of the drying chamber.

The apparatus further comprises a temperature sensor configured to monitor a temperature of the saturation chamber. Non-limiting examples of temperature sensor include thermometer, resistance temperature detector (RTD), thermistor, etc. In some embodiments, the temperature sensor is a mechanical temperature gauge/thermometer to measure the saturation temperature rather than an electronic method which would require hazardous area certifications for use in explosive atmospheres.

In some embodiments, the saturation chamber comprises an absorbent material that is wetted with water to provide large surface area for water to evaporate and saturate the first portion gas stream flowing through the saturation chamber. Non limiting examples of suitable absorbent material include a sponge, a cotton wool, a cellulose fiber, etc.

The saturation chamber can have a volume to provide sufficient residence time for the first portion stream being saturated. The residence time is determined according to the range of absolute pressures being used, the ambient temperature range where the apparatus is used, the moisture content range associated with the desired moisture content, and/or the required outlet flow rate range.

In some embodiments, the located apparatus is located in a temperature controlled environment, such as an enclosure, building, etc. The temperature controlled environment can include a temperature regulator to control the temperature of the temperature controlled environment.

Regulation of the temperature of the temperature controlled environment can be done in proportion to the temperature measured for the saturation chamber by the temperature sensor. In other words, the temperature of the apparatus itself can be regulated according to the temperature measured for the saturation chamber.

In some embodiments, the drying chamber comprises a desiccant. Non limiting examples of suitable desiccant include alumina, phosphorus pentoxide, silica gel, activated charcoal, calcium sulfate, calcium chloride, and molecular sieves (typically zeolites), etc.

The water vapor concentration in the calibrated moisture-gas mixture generated by the apparatus of the present disclosure can be calculated by determining the vapor pressure of water at the temperature of the saturation chamber, dividing this pressure by the absolute pressure in the saturation chamber to determine the molar fraction of water, and reducing this molar fraction by the dilution factor created by the two critical flow orifices. The vapor pressure of water at the temperature of the saturation chamber can be calculated using a suitable method, such as the Golf Gratch method, Hyland and Wexler method, Buck method, Sontag method, or Magnus Tetens method.

In one embodiment, the apparatus has two fixed critical flow orifices as the first and second flow control devices. In these embodiments, the dilution factor used in the calculation can be calculated using the equation (1) and using either gas flow rates calculated from the orifice sizes at a reference inlet pressure for a reference gas, flow rates measured by the orifice manufacturer at a reference inlet pressure for a reference gas, or measured flow rates for both orifices measured for the actual gas used in the application.

When critical orifices are used as the flow control devices, the minimum ratio of the pressure of the pressurized gas stream and the outlet pressure of the moisture/gas mixture can be calculated according to the following equation:

$${P}_{I} / {P}_{O} = {( [ γ + 1 ] / 2 )}^{γ / ( γ - 1 )} \qquad Eq . 2$$

wherein:
P_{I}=Absolute pressure at orifice inlet
P_{O}=Absolute pressure at orifice outlet
γ=Ratio of specific heats Cₚ/Cᵥ for the gas, (i.e. heat capacity ratio of specific heats at constant pressure Cₚ and at constant volume Cᵥ for the gas.
For air γ=1.40 @20 C, for methane γ=1.307 @ 20 C, for helium γ=1.66 @ 20 C.

In some embodiments, additional flow control devices and/or selection valves are included in parallel with the first and second flow control devices to provide additional adjustability.

The calculation of the resulting water vapor concentration in the calibration gas-mixture is therefore based on a measured but not controlled temperature, a measured and adjustable gas pressure, and a fixed dilution ratio.

In some embodiments, the apparatus includes a programmable logic computer that is configured to obtain measurements of control variables, such as pressure of the pressurized gas stream and temperature of the saturation temperature. The programmable logic computer can be further configured to execute calculations for achieving the desired moisture content in the calibration moisture-gas mixture. The programmable logic computer can be further configured to direct regulation components of the apparatus, such as the electronic pressure controller, and/or the temperature regulator, in accordance with one or more of the control variables and/or the calculations, to achieve the desired moisture content.

The programmable logic computer can be an electronic device that includes a processor, such as a central processing unit (CPU), memory, and a bi-directional bus to communicatively couple the components of programmable logic computer. In some embodiments, the programmable logic computer further includes non-transitory mass storage, a network interface, an I/O interface, and/or a transceiver. The memory can include any type of tangible, non-transitory memory such as static random access memory (SRAM), dynamic random access memory (DRAM), synchronous DRAM (SDRAM), read-only memory (ROM), any combination of such, or the like. The mass storage element can include any type of tangible, non-transitory storage device, such as a solid state drive, hard disk drive, a magnetic disk drive, an optical disk drive, USB drive, or any computer program product configured to store data and machine executable program code. According to certain embodiments, the memory and/or mass storage can have recorded thereon statements and instructions executable by the processor for performing any of the method operations described herein, such as calculations for achieving the desired moisture content. According to certain embodiments, any or all of the depicted elements may be utilized, or only a subset of the elements. Further, the programmable logic computer contain multiple instances of certain elements, such as multiple processors, memories, or transceivers. Additionally or alternatively to a processor and memory, other electronics, such as integrated circuits, can be employed for performing the required logical operations of the programmable logic computer.

In some embodiments, the apparatus of the present disclosure enables a user to define a set of moisture-gas mixture setpoints or a corresponding set of moisture content setpoints. The programmable logic computer can receive the set of moisture-gas mixture setpoints or the set of moisture content setpoints through an I/O interface. In embodiments, the desired moisture content of the moisture-gas mixture achieved by the apparatus can be one of the set of moisture content setpoints received from the user. In some embodiments, the apparatus can be configured to achieve the set of moisture content setpoints or the set of moisture-gas mixture setpoints through a sequential series of moisture-gas mixtures.

In some implementations, the back-pressure regulator positioned near the outlet of the apparatus can be used to control the pressure at the outlet to vent excess gas flow when the total flow of the calibration moisture-gas mixture exceeds the flow capacity needs of the moisture analyzer being tested or when a certain delivery pressure for the calibration moisture-gas mixture is required. If critical flow orifices are used as the flow control devices, the back-pressure regulator near the outlet of the apparatus can be used to control the pressure on the outlet of the critical flow orifices to ensure that the inlet to outlet pressure ratio is high enough for the orifices to be critical (i.e., the flow velocity is at the local speed of sound). In some embodiments, the back-pressure regulator can be directed by the programmable logic computer to control the pressure at the outlet.

In some implementations, the first flow control device can be on the inlet to the saturation chamber and an additional back-pressure regulator can be used on the outlet of the saturation chamber to control the saturation pressure and thereby adjust the water vapor content in the calibration moisture-gas mixture.

To gain a better understanding of the present disclosure described herein, the following example is set forth with reference to the accompanying drawing, which is not drawn to scale, and the illustrated components are not necessarily drawn proportionately to one another. It will be understood that this example is intended to describe an illustrative embodiment of the present disclosure and is not intended to limit the scope of the present disclosure in any way.

### Examples

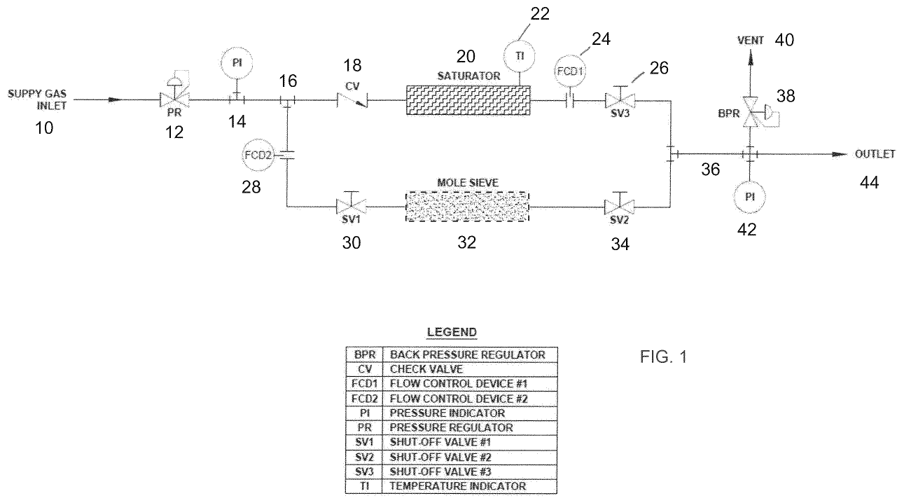

EXAMPLE 1: FIG. 1 depicts a general flow diagram illustrating an exemplary apparatus for conducting methods of the present disclosure.

Referring to FIG. 1, the apparatus has an inlet (10) configured for connection to a supply of gas at high pressure to receive a pressurized gas stream, followed by an optional pressure regulator (12) configured to regulate the pressure of the pressurized gas stream to a desired value (for example, in the case where the gas supply pressure is not very stable or if it is desirable to adjust the moisture content of the generated gas mixture). A first pressure indicator (14) such as a pressure gauge, transmitter, or transducer, is placed downstream of the pressure regulator (12) to measure and monitor the pressure. A splitter (16) is positioned downstream of the pressure regulator (12) to separate the pressurized gas stream into a first portion stream and a second portion stream.

The apparatus of FIG. 1 further comprises a saturation chamber (20) configured to receive the first portion stream to form a water-saturated gas stream therefrom, and an optional drying chamber (32) configured to receive the second portion stream to form a dry-gas stream therefrom.

A first flow control device (24) is located downstream of the saturation chamber (20), which is configured to regulate flow of the water-saturated gas stream flowing out from the saturation chamber (20), and a second flow control device (28) is positioned upstream (or downstream) of the drying chamber (32), which is configured to regulate flow of the second portion stream.

In alternative embodiments (not shown), the first flow control device (24) can be located upstream of the saturation chamber (20); in these cases, the pressure of the water-saturated gas stream would be controlled by an additional back pressure regulator located at the outlet of the saturation chamber (20).

A mixing chamber (36) in the form of a tube or pipe of a desired length is configured to receive and mix the water-saturated gas stream from the saturation chamber (20) and the second portion stream through optional drying chamber (32) to form the calibration moisture-gas mixture, which is released via an outlet (44).

The saturation chamber (20) is filled with an absorbent material wetted with water to provide a surface area for water to evaporate and saturate the gas flowing through the chamber. The volume of the chamber is chosen to provide adequate residence time taking into account the range of absolute pressures being used, the ambient temperature range where the apparatus will be used, the target water content range, and the range of moisture/gas mixture flow being generated.

The apparatus of FIG. 1 is further provided with a check valve (18) upstream of the saturation chamber to prevent back-flow or diffusion of moisture from the saturation chamber (20) to the inlet (10). The apparatus is further provided with an optional back-pressure regulator (38) and a second pressure indicator (42), positioned between the mixing member (36) and the outlet (44) to monitor and regulate the pressure of the calibration moisture-gas mixture exiting the outlet (44). Optionally, a vent member (40) is connected to the back-pressure regulator (38) to vent excess gas flow that exceeds the flow capacity of the moisture analyzer being tested.

A first selection/shut-off valve (26) is provided downstream of the first flow control device (24), a second selection/shut-off valve (30) is provided upstream the drying chamber and/or a selection/third shut-off valve (34) is provided downstream the drying chamber (32).

For the generation of a calibration moisture-gas mixture, the pressurized gas stream received at the inlet (10) flows towards the splitter through the optional pressure regulator (12), at a known pressure and is divided into a first portion stream and a second portion stream via the splitter (16). The first portion stream flows through the optional check valve (18) into the saturation chamber (20) at the known and/or a target/desired pressure, to become a water-saturated gas stream, which then flows through the first flow control device (24). The second portion stream flows through the second flow control device (28) through the optional drying chamber (32). As shown in FIG. 1, pressure indicator (14) is located before the splitter. Alternatively, the pressure indicator can be located before, after, or both before and after the check valve (18), but before the first flow control device (24). The temperature of the saturation chamber (20) is monitored by the temperature indicator (22). If required, the pressure of the pressurized gas stream received from the gas supply is regulated by pressure regulator (12) to achieve the target/desired pressure to control the water saturator contact pressure, the produced water vapor content, and/or the total system flow rate.

The water-saturated gas stream flows out of the saturation chamber (20), through the first flow control device (24), and is then mixed with the second portion stream.

In the present example, the first flow control device (24) and the second flow control device (28) are “critical flow orifices”. The two orifice sizes are selected to regulate the flow of the water-saturated gas stream and the second portion stream to achieve a desired constant flow ratio between these streams, which provides a suitable dilution factor for achieving the desired moisture content range in the calibration moisture-gas mixture.

To validate the accuracy of analyzers measuring moisture in natural gas where the pressure range typically extends from around 1000 kPag (145 psig) to 6900 kPag (1000 psig), dilution factors between 1:10 and 1:25 can work well, but other dilution factors can be used.

After mixing in the mixing member (36), the resulting calibration moisture-gas mixture flows out of the outlet (44). The pressure at the outlet (44) is monitored with the second pressure indicator (42) and can be regulated via the optional back-pressure regulator (38) connected to the vent (40). The back-pressure regulator (38) is installed sufficiently far downstream of the mixing member (36) to ensure that the resulting calibration moisture-gas mixture is homogenous before any excess flow is vented through the back-pressure regulator (38). An excess flow that is not consumed by any devices is vented.

The water vapor concentration in the calibration moisture-gas mixture generated is calculated by determining the vapor pressure of water at the saturation chamber temperature and then dividing this pressure by the absolute pressure in the saturation chamber to determine the mole fraction of water and then reducing this molar fraction by the dilution factor created by the two flow orifices.

In the simplest operation of apparatus of the present example, the temperature of the saturation chamber and the rest of the apparatus is not controlled, but only measured as this arrangement lowers the size and cost and avoids the need for additional utilities such as electrical power. The adjustment of the water vapor content in the water-saturated gas mixture is achieved by using optional inlet pressure regulator to vary the total absolute pressure in the saturation chamber, thereby changing the water vapor content of the gas flowing through flow orifice (24).

The simplest version of this apparatus has only two fixed critical flow orifices to control both the dry gas and water saturated gas flows as this arrangement has minimum size and cost, while simplifying the operation of the apparatus. The flow through critical flow orifices is stable, accurate and repeatable for long periods of time so employing them in this application avoids the need to periodic calibration that other methods of adjustable flow control require (e.g. metering valves combined with flow meters or electronic mass flow controllers). These critical flow orifices also allow the apparatus to operate accurately over a very wide range of flow rates and pressures while being unaffected by feed gas composition and outlet pressure variations.

Although a particular embodiment of the present disclosure has been illustrated and described, the scope of the claims should not be limited by the embodiment set forth in the above example or drawing, but should be given the broadest interpretation consistent with the description as a whole.

What is claimed:

1. An apparatus for producing a calibration moisture-gas mixture for validation testing of moisture analyzers, the apparatus comprising:
an inlet configured to receive a pressurized gas stream from a gas supply;
a pressure indicator to measure pressure of the pressurized gas stream from the gas supply;
a splitter configured to separate the pressurized gas stream into a first portion stream and a second portion stream;
a saturation chamber configured to receive the first portion stream, the saturation chamber containing a water component for generating water vapor for saturating the first portion stream form a water-saturated gas stream;
a temperature sensor configured to measure temperature of the saturation chamber;
a first flow control device positioned upstream or downstream of the saturation chamber and configured to regulate flow of the water-saturated gas stream flowing through/out from the saturation chamber;
a second flow control device positioned downstream of the splitter and configured to regulate flow of the second portion stream;
a mixing member and an outlet;
the mixing member being configured to receive and mix the water-saturated gas stream and the second portion stream to form the calibration moisture-gas mixture while moving the calibration moisture-gas mixture towards the outlet
the outlet configured to output the calibration moisture-gas mixture; and
wherein the first control device and second flow control device are configured to achieve a known constant flow ratio between the second portion stream and the water-saturated gas stream to provide a constant predefined dilution factor at the measured temperature of the saturation chamber and the measured pressure of pressurized gas stream without regulating the temperature of the saturation chamber, to form the calibration moisture-gas mixture with a desired moisture content.

2. The apparatus of claim 1, further comprising a pressure regulator upstream of the splitter configured to regulate pressure of the pressurized gas stream before the pressurized gas stream is separated into the first portion stream and the second portion stream.

3. The apparatus of claim 1, wherein the first flow control device and the second flow control device are independently selected from a critical flow orifice, a porous metal device.

4. The apparatus of claim 1, further comprising a drying chamber configured to receive the second portion stream and form a dry-gas stream therefrom.

5. The apparatus of claim 4 wherein the second flow control device is positioned upstream or downstream of the drying chamber.

6. The apparatus of claim 4, wherein the drying chamber comprises a desiccant.

7. The apparatus of claim 1, further comprising a check valve positioned upstream of the saturation chamber.

8. The apparatus of claim 1 wherein the water component of the saturation chamber is an absorbent material wetted with water.

9. The apparatus of claim 1, further comprising:
an additional pressure indicator configured to monitor pressure of the calibration moisture-gas mixture; and
a back-pressure regulator configured to regulate pressure of the calibration moisture-gas mixture.

10. The apparatus of claim 9, further comprising:
a vent member connected to the back-pressure regulator and configured to vent an excess gas flow exceeding flow capacity of the moisture analyzer being tested.

11. The apparatus of claim 1, wherein the pressurized gas stream includes natural gas, nitrogen, hydrogen, sour gas, or a mixture thereof.

12. The apparatus of claim 1, further comprising one or more additional flow control devices in parallel with the first and second flow control devices to provide additional adjustability.

13. A method for producing a calibration moisture-gas mixture for validation testing of moisture analyzers, the method comprising:
receiving a pressurized gas stream from a gas supply and measuring the pressure thereof;
separating the pressurized gas stream into a first portion stream and a second portion stream;
saturating the first portion stream with water vapor in a saturation chamber containing a water component, to form a water-saturated gas stream, at a known temperature of the saturation chamber;
controlling flow rate of the water-saturated gas stream and the second portion stream to achieve a constant predefined flow rate ratio between the second portion stream and the water-saturated gas stream, without regulating the temperature of the saturation chamber; and
mixing the water saturated-gas stream and the second portion stream to form the calibration moisture-gas mixture with a constant predefined dilution factor to obtain the calibration moisture-gas mixture with a target moisture content.

14. The method of claim 13, further comprising regulating pressure of the pressurized gas stream before the separating step or regulating pressures of the water saturated-gas stream and the second portion stream in accordance with the temperature of the saturation chamber.

15. The method of claim 13, wherein the constant predefined dilution factor is defined by a ratio of a sum of the flow rate of the first portion stream and the flow rate of the second portion stream, and the flow rate of the water-saturated gas stream, calculated using the equation below:
$$DF = ( FO 1 + FO 2 ) / FO 1$$
wherein: DF=dilution factor (expressed as 1:DF);
FO1=flow rate of the water-saturated gas stream; and
FO2=flow rate of the second portion stream.

16. The method of claim 13, wherein the constant predefined flow rate ratio between the second portion stream and the water-saturated gas stream is at least 1.5:1.

17. The method of claim 13, wherein the water component is an absorbent material wetted with water.

18. The method of claim 13, further comprising drying the second portion stream to form a dry-gas stream.

19. The method of claim 18, comprising controlling the flow of the second portion stream before or after the drying step.

20. The method of claim 18, wherein method comprises passing the second portion stream through a drying chamber comprising a desiccant.

21. The method of claim 18, wherein the flow rates of the water-saturated gas stream, and the second portion stream is controlled via using at least one of a critical flow orifice, a porous metal device.

22. The method of claim 13, wherein the pressurized gas stream includes at least one of natural gas, carbon dioxide, nitrogen, hydrogen, and sour gas.

23. The apparatus of claim 1 further comprising an electronic pressure controller configured to regulate pressure of the pressurized gas stream.

24. The apparatus of claim 1 located in a temperature controlled environment including a temperature regulator.

25. The apparatus of claim 23 further comprising a programmable logic computer configured to:
obtain, from the temperature sensor, the measured temperature of the saturation chamber; and
direct the electronic pressure controller to regulate the pressure of the pressurized gas-stream.

26. The apparatus of claim 24 further comprising a programmable logic computer configured to:
obtain, from the temperature sensor, the measured temperature of the saturation chamber; and
direct the temperature regulator in accordance with the measured temperature of the saturation chamber.

27. The apparatus of claim 25 wherein the programmable logic computer is configured to receive a set of moisture content setpoints; and
the desired moisture content is one of the set of moisture content setpoints.
